(19) United States
(12) Patent Application Publication
(10) Pub. No.: US 2009/0162417 A1
(43) Pub. Date: Jun. 25, 2009

(54) DRUG ELUTING OCULAR CONFORMER

(75) Inventor: Scott E. Eells, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/337,369

(22) Filed: Dec. 17, 2008

Related U.S. Application Data
(60) Provisional application No. 61/016,291, filed on Dec. 21, 2007.

(51) Int. Cl. A61F 2/16

(52) U.S. Cl. 424/427

(57) ABSTRACT

A coated, drug eluting ocular conformer includes an ocular conformer and at least one substantially purified anti-fibrosis agent. The ocular conformer is formed from a base material having inner and outer sides, including apical and basal portions configured to contact one or more conjunctival tissues in an eye of a patient. The anti-fibrosis agent is formulated into at least one ophthalmic medicament layer over at least one side of the ocular conformer or is impregnated within the base material of the ocular conformer. The device may be configured to release the anti-fibrosis agent from one or both sides of the ocular conformer. An elution control layer may be included to facilitate controlled release of the anti-fibrosis agent. In addition, an adhesion promoting layer may be included in the device to promote adhesion of polymeric layers to the base material or to ocular tissues during delivery. The coated, drug eluting ocular conformer may be used to reduce scarring in the eye, typically by applying the device to the eye following eye surgery, an eye injury caused by chemical, thermal or mechanical trauma, or an eye disease or condition associated with scarring.

This application claims the benefit of priority under 35 U.S.C. § 119(e) to U.S. Provisional Application No. 61/016,291, filed Dec. 21, 2007, which is hereby incorporated by reference in its entirety.

### TECHNICAL FIELD

The present invention relates to a modified prosthetic device for localized delivery of medicaments to the eye, particularly with regard to reduction or prevention of scarring.

### BACKGROUND

There are various postoperative complications and disease conditions in the field of opthalmology. Many of these complications relate to wound healing processes in the eye following surgery, following chemical, thermal, or traumatic injuries, or those associated with ophthalmic diseases or disease conditions. For example, extensive scar formation (cicatrix) of the conjunctiva can be found in a number of severe cicatricial ocular surface diseases, including multirecurrent ptergyia, proliferative vitreoretinopathy (PVR), Stevens-Johnson syndrome (SJS), and ocular cicatricial pemphigoid (OIP).

Glaucoma is a condition characterized by increased intraocular pressure in the eye which, if left untreated, leads to blindness. The increase in the intraocular pressure can be initially treated with medications but eventually may need surgery to lower the pressure in the eye to prevent blindness. The common surgery performed is glaucoma filtration surgery. The most common cause of failure of glaucoma filtration surgery is scarring, typically by proliferation cells (fibroblasts) and fibrosis (scarring) in the subconjunctival space under the surface covering of the eye. Scarring can lead to an increase in intraocular pressure.

The wound healing process involves a delicate balance between the synthesis and degradation of connective tissue matrix components. Inappropriate scarring can result from an unbalanced wound healing process being run amok. This can lead to many of the above-described ophthalmic complications.

The wound healing process can be can be modulated through several approaches, including inhibiting proliferation or migration of fibroblasts, inhibiting inflammation or the acute inflammatory response, inhibiting angiogenesis, reducing extracellular matrix (ECM) production or promoting ECM breakdown, and/or inhibiting tissue remodeling.

Therapeutic agents for inhibiting scarring are known. Several agents including anti-neoplastic agents (cancer chemotherapy) and corticosteroids have been studied experimentally, both in vivo and in vitro. Commonly administered ophthalmic agents include antiproliferative agents, such as mitomycin-C and 5-fluorouracil, have been widely used to modulate the wound healing process, particularly following surgery, such as glaucoma filtration surgery. However, 5-fluorouracil has the drawback of frequent post-operative subconjunctival injections and is associated with complications, such as wound leakage and corneal epithelial defects. Mitomycin-C is conveniently administered in a single application at the time of surgery but its use is associated with several complications, including excessive filtration, and can result in persistently low intraocular pressure and associated problems, including decreased vision or blurred vision.

Such complications appear to be attributable, at least in part, to the current non-specific, uncontrolled delivery methods for administering these drugs. Typically, their administration is initially accompanied by high drug concentrations that are not appropriately sustained, nor well tolerated as reflected in the various ocular toxicities or pathological complications that may follow. Thus there is a demand for new therapies or modes of ocular delivery which will reduce scarring in the eye, and increase the success rate of ophthalmic surgeries (such as glaucoma filtration surgery), while avoiding the complications seen with 5-fluorouracil and mitomycin-C.

Conventional procedures employing topical ophthalmic medicament applications are limited in their effectiveness as most of the medicament is lost due to run-off on account of the contour of the eye and eyelids. This limits the beneficial amount of contact time between agents and the ocular surfaces.

Methods for drug delivery to the eye have been described. U.S. Pat. No. 4,240,163 describes an intraocular lens coated with a compatible medicament, such as an anticoagulant, an anti-inflammatory agent or an anti-complement agent. Application of the coated lens is invasive, requiring surgical implantation.

Attempts have been made to relieve the limitations of topical delivery through systems providing sustained drug release to the eye. Prior topical sustained release systems include gradual release formulations, either in solution or ointment form, which are applied to the eye in the same manner as eye drops but less frequently. Such formulations are disclosed, for example, in U.S. Pat. No. 3,826,258 issued to Abraham and U.S. Pat. No. 4,923,699 issued to Kaufman. Due to their method of application, however, these formulations result in many of the same problems detailed above for conventional eye drops. In the case of ointment preparations, additional problems are encountered such as a blurring effect on vision and the discomfort of the sticky sensation caused by the thick ointment base.

Alternatively, sustained release systems have been configured to be placed into the conjunctival cul-de-sac, between the lower lid and the eye. Such units typically contain a core drug-containing reservoir surrounded by a hydrophobic copolymer membrane which controls the diffusion of the drug. Examples of such devices are disclosed in U.S. Pat. Nos. 3,618,604 and 3,828,777, issued to Ness, U.S. Pat. No. 3,626,940, issued to Zaffaroni, U.S. Pat. No. 3,845,770, issued to Theeuwes et al., U.S. Pat. No. 3,962,414, issued to Michaels, U.S. Pat. No. 3,993,071, issued to Higuchi et al., and U.S. Pat. No. 4,014,335 issued to Arnold. However, due to their architectures, many of these devices are sub-optimal with regard to comfort, movement and sensation within the formix felt by the patient, and general irritation resulting in less than adequate patient acceptance.

Other controlled release devices require surgical implantation, sub-optimal placement requirements, or are osmotically driven wherein an osmotic or ionic gradient responsible for the drug efflux from the device. This may necessitate additional osmotic or ionic agents, which may not be compatible with the ocular environment. Thus, there is a need for a noninvasive drug delivery device that is simple in design, easy to apply, and does not require an osmotic or ionic agent for drug efflux and yet accomplishes the objectives of prolonged and uninterrupted ocular drug delivery.

An ocular conformer is a device made of molded plastic fitted in the space between the eyeball and eyelid to maintain space in the orbital cavity, prevent socket contraction, and prevent closure and/or adhesions between the eyeball and the eyelid during the healing process following surgery. Ocular conformers are generally small concave devices having an inner surface shaped to approximately match the curvature of the orbit. Ocular conformers are frequently used by oculoplastic surgeons at the end of reconstructive surgery to prevent the postoperative formation of symblepharon, i.e., fibrotic adhesion between the tarsal conjunctiva of the eyelid and the bulbar conjunctiva of the globe. Unlike other devices for ocular drug delivery, ocular conformers are noninvasive and can be applied to the ocular surface without sutures.

WO 2006/093370 is directed to an artificial eye and conformer, which are produced by a process comprising addition of materials having antibacterial and bactericidal activities to acrylic resin powder, including loess, zeolite, bentonite, bioceramic or nano-silver, so that the artificial eye and the conformer have antibacterial activity in themselves.

US 2004/0181240 disclose a “bandage contact lens” device, in which an amniotic membrane covering is fitted over a conformer ring structure fitted in the space between the eyelids and the ocular surface or cornea. The amniotic membrane forms a covering over the entirety of the corneal or ocular surface and is designed to protect corneal tissue, prevent adhesions, exclude bacteria, inhibit bacterial activity, and promote healing and tissue remodeling. In addition, therapeutic agents can be incorporated into the amniotic membrane covering or into the ring-based support structure, thereby serving as a controlled release drug delivery vehicle.

In view of the above problems and limitations, there is a need in the art for improved compositions and methods for drug delivery to the eye. The drug-eluting ocular conformer according to the present invention offers significant advantages over conventional materials and methods by increasing contact time and efficiency of ophthalmic medicament release to ocular surfaces. Further, it is believed that the compositions and methods described below can be provide a more tolerable alternative to painful and invasive injections or drugs according to prior art applications, including, for example, intravitreal steroid injections for treatment of diabetic retinopathy, retinal vascular occlusions, and wet, age-related macular degeneration.

### SUMMARY

In one aspect, a coated, drug eluting ocular conformer includes an ocular conformer and at least one substantially purified anti-fibrosis agent. The ocular conformer is formed from a base material having inner and outer sides, including apical and basal portions configured to contact one or more conjunctival tissues in an eye. The anti-fibrosis agent is formulated into at least one ophthalmic medicament layer over at least one side of the ocular conformer or is impregnated within the base material of the ocular conformer. The device may be configured to release the anti-fibrosis agent from one or more one or both sides of the ocular conformer. An elution control layer may be included to facilitate controlled release of the anti-fibrosis agent. The elution control layer may be posited over the medicament layer or over an ocular conformer base material impregnated with the anti-fibrosis agent. In addition, an adhesion promoting layer may be included in the device to promote adhesion of polymeric layers to the base material or to ocular tissues during delivery.

In another aspect, a method for making a coated, drug eluting ocular conformer device includes providing an ocular conformer and incorporating into the device a substantially purified anti-fibrosis. The ocular conformer is formed from a base material and includes an inner side and an outer side, the inner side being configured to contact an eye. The anti-fibrosis agent is impregnated within the base material of the ocular conformer or is formulated into an ophthalmic medicament layer that is posited on one side or both sides of the ocular conformer. Additional layers, including an elution control layer and an adhesion control layer may be further included in the device to facilitate controlled delivery of the anti-fibrosis agent.

In a further aspect, a method for method for reducing scarring in the eye includes applying to an eye of a subject a coated, drug eluting ocular conformer according to the present invention. The coated device is typically applied the eye following eye surgery, an eye injury caused by chemical, thermal or mechanical trauma, or an eye disease or condition associated with scarring.

### BRIEF DESCRIPTION OF THE DRAWINGS

FIG. 1 is a side sectional view illustrating the anterior portion of the human eye.

FIG. 2A is a schematic illustration of an exemplary coated drug eluting ocular conformer device according to one embodiment of the present invention.

FIG. 2B is a cross-sectional view of the coated drug eluting ocular conformer device depicted in FIG. 2A.

FIG. 3A is a cross-sectional view of an exemplary coated drug eluting ocular conformer device according to another embodiment of the present invention.

FIG. 3B is a cross-sectional view of an alternative multi-layered coating relative to that depicted in FIG. 3A.

FIG. 4 is a cross-sectional view of an exemplary coated drug eluting ocular conformer device according to a further embodiment of the present invention.

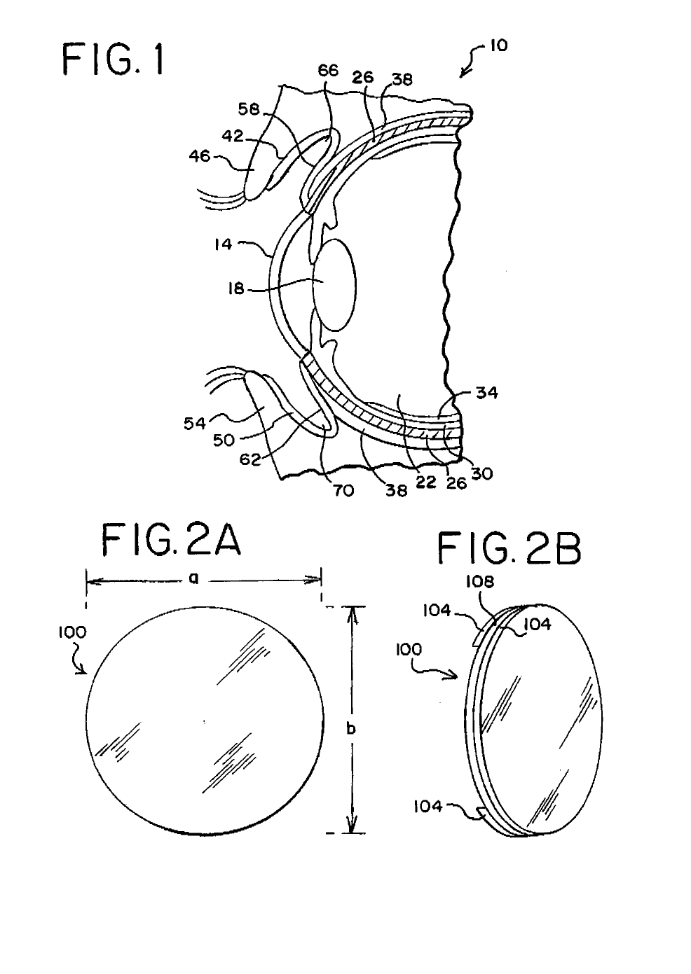

The various elements depicted in the drawings are merely representational and are not drawn to scale. For example, the proportions and thicknesses of the various layers, coatings or tissue portions in FIGS. 1-4 have been exaggerated in some instances or minimized in others. The drawings are intended to illustrate various implementations of the invention, which can be understood and appropriately carried out by those of ordinary skill in the art.

### DETAILED DESCRIPTION

### Definitions of Terms

It is to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein may be used in the practice or testing of the invention, the preferred methods, devices and materials are now described.

As used herein, the terms “medicament”, “therapeutic agent” and “drug” are used interchangeably to designate an organic compound, inorganic compound, biological polymer, peptide, polypeptide, antibody, peptide conjugate, nucleic acid, oligonucleotide, polynucleotide, ribozyme, or small interfering RNA (siRNA) capable of rendering a beneficial physiological effect in treating a pathological condition when administered alone or in combination with other active agents or drugs to a subject in need of such treatment. The medicament, therapeutic agent or drug is capable of modifying, controlling, delaying or reversing a disease or disorder or ameliorating the symptoms of a disease or disorder in a subject.

The term “ocular conformer” refers to an art-recognized concave medical prosthetic device with an elliptically shaped planar circular surface structure having an inner surface generally conforming to the curvature and contours of the orbit, covering the cornea and extending into the upper and lower formices surrounding the upper and lower eyelid. As used herein, the ocular conformer is configured to prevent closure and/or adhesions between the orbit and eyelid during the post surgical healing process, and to form a temporary replacement for a custom-designed cosmetic artificial eye molded to fit between the eyelids over the conjunctiva covering an orbital implant.

The terms “fibrosis,” “scarring,” or “fibrotic response” are used interchangeably to refer to fibrous (scar) tissue formation in response to injury or medical intervention.

The terms “inhibit fibrosis”, “reduce fibrosis”, “inhibits scarring” and the like are used synonymously to refer to the action of agents or compositions which result in a statistically significant decrease in the formation of fibrous tissue that can be expected to occur in the absence of the agent or composition.

The terms “anti-fibrosis agent”, “fibrosis-inhibiting agent”, “fibrosis-inhibitor”, and “anti-scarring agents” are used interchangeably to designate a therapeutic agent inhibiting fibrosis or scarring through one or more of the following mechanisms including: inhibiting proliferation or migration of fibroblasts, inhibiting inflammation or the acute inflammatory response, inhibiting angiogenesis, reducing extracellular matrix (ECM) production or promoting ECM breakdown, and/or inhibiting tissue remodeling.

As used herein, the term “antiproliferative agent” refers to a compound that inhibits the growth of fibroblasts. Antiproliferative agents include, but are not limited to microtubule inhibitors, topoisomerase inhibitors, platins, alkylating agents, and anti-metabolites. Exemplary antiproliferative agents taxane compounds and analogues, including paclitaxel, docetaxel, ABRAXANE; 5-fluorouracil, mitomycin C, gemcitabine, doxorubicin, vinblastine, etoposide, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, estramustine phosphate, etoposide, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitotane, mitoxantrone, pentostatin, procarbazine, rituximab, streptozocin, tamoxifen, temozolomide, teniposide, 6-thioguanine, topotecan, trastuzumab, vincristine, vindesine, and vinorelbine.

The term “antibiotic” is art recognized and includes antimicrobial agents synthesized by an organism, isolated from the natural source, and includes natural or chemically synthesized analogs thereof.

The term “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity

The terms “patient,” “subject,” and “recipient” as used in this application refer to any mammal, especially humans. For purposes of treatment, the term “mammal” refers to any animal classified as a mammal, including humans, domestic and farm animals, and zoo, sports, or pet animals, such as dogs, horses, cats, cattle, pigs, sheep, etc.

### Drug Eluting Ocular Conformer Configurations

In one aspect, a drug eluting ocular conformer device includes an ocular conformer base material eluting an ophthalmic medicament. The ophthalmic medicament may be posited directly on at least a portion of the ocular conformer base material or it may be posited in or over a coating layer posited over the base material. The medicament layer(s) and/or additional coating layers associated therewith may be posited one or both sides of the base material. Accordingly, the device is defined by one or more ophthalmic medicament layers from which controlled release of the ophthalmic medicament is achieved.

The drug eluting ocular conformer device may further include an elution control layer posited over one or more ophthalmic medicament layer(s) and/or coating layers; the elution control layer is composed of polymeric material providing for controlled release of the ophthalmic medicament through the elution control layer. In one embodiment, the elution control layer is defined by a porous layer of polymeric material of defined thickness chosen to provide a desired level of controlled release. In one embodiment, the elution control layer is defined by a bioabsorbable elastomer layer.

In a further aspect, the ocular conformer device of the present invention may further include at least one adhesion promoting layer. An adhesion promoting layer can be utilized to facilitate adhesion of the ophthalmic medicament layer to the base material of the ocular conformer, to the ocular surface, or both.

FIG. 1 depicts a side sectional view schematically illustrating the human eye 10. The eye 10 has a cornea 14, lens 18, vitreous 22, sclera 26, choroid 30, and retina 34. The Tenon's membrane or Tenon's capsule 38 is disposed on the sclera 26. The upper palpebral conjunctiva 42 covers the posterior surface of the upper eyelid 46 and the lower palpebral conjunctiva 50 covers the posterior surface of the lower eyelid 54. The upper bulbar conjunctiva 58 and the lower bulbar conjunctiva 62 are each disposed on the Tenon's capsule 38. The upper palpebral conjunctiva 42 folds up into the upper bulbar conjunctiva 58 to form an upper cul-de-sac or upper formix 66. The lower palpebral conjunctiva 50 folds down into the lower bulbar conjunctiva 62 to form a lower cul-de-sac or lower formix 70.

With reference now to FIGS. 2A and 2B, a coated, drug-eluting ocular conformer 100 includes an ophthalmic medicament layer 104 posited over the inner surface of an ocular conformer 108. Ophthalmic medicament layer 104 portions are terminally posited onto the outer surface of the ocular conformer 108 and over the entire inner surface of the ocular conformer 108.

One of skill in the art will appreciate that an ocular conformer 108 is a commercially available prosthetic medical device (Porex Surgical Products, Newnan, Ga.), which is configured to prevent closure and/or adhesions between the orbit and eyelid during the post surgical healing process, and to form a temporary replacement for a custom-designed cosmetic artificial eye molded to fit between the eyelids over the conjunctiva covering an orbital implant. An ocular conformer is used in the latter to hold the eyelids in place, prevents shrinkage of the space between the inner surface of the eyelids and the conjunctival covering of the orbital implant, and allow for healing in the surrounding socket tissues. Ocular conformers are available in vented or non-vented configurations.

The ocular conformer 108 is concave, with an elliptically shaped planar circular surface structure (FIG. 2A) having an inner surface generally conforming to the curvature and contours of the orbit, covering the cornea and extending into the upper and lower formices surrounding the upper and lower eyelids. In order to extend into upper formix 66 on one side and the lower formix 70 on the other, the ocular conformer 108 will typically have a diameter of no less than 18 mm. In particular, the ocular conformer 108 will preferably have a diameter a between about 20 mm and 26 mm, and a diameter b between about 18 mm and 24 mm. In addition, the ocular conformer 108 will generally have a thickness between about 3 mm to about 8 mm, preferably between about 4 mm to about 6 mm.

A coated, drug eluting ocular conformer device according to the present invention may alternatively employ a symblepharon ring as a support or coating substrate in place of the above-described ocular conformer 108. A symblepharon ring can be commercially obtained in various sizes (Jardon Eye Prosthetics Inc., Southfield, Mich.) for use in postoperative symblepharon repair. Like the ocular conformer above, the symblepharon ring extends into the upper and lower formices 66, 70 under the upper and lower eyelids 46, 54. However, in contrast to the ocular conformer above, the symblepharon ring contains a central opening generally corresponding to the area overlaying the cornea 14.

Ocular conformer and symblepharon ring base materials are generally formed from acrylic materials, such as polymethylmethacrylate, or other suitable materials, such as lucite and silicon.

FIG. 2B shows a sided cross-section of the coated drug eluting ocular conformer device 100 in FIG. 2A illustrating ophthalmic medicament layer 104 portions terminally posited onto the outer surface of the ocular conformer 108 and over the entire inner surface of the ocular conformer 108. Ophthalmic medicament layer portions 104 are further posited over the terminal outer surfaces of the ocular conformer 108. Ophthalmic medicament layer(s) 104 may be formed over the entire inner and/or outer surface(s) of the ocular conformer 108, or they may be selectively targeted to one or more places on one side or both sides of the ocular confirmer 108 as illustrated in FIG. 2B. Preferably, when used for delivering anti-fibrosis agents, ophthalmic medicament layer(s) 104 will be configured for placement adjacent to one or more of the conjunctival tissues 42, 50, 58, 62 in the upper and lower formices 66, 70. Thus, as illustrated in FIG. 2B, ophthalmic medicament layers 104 may be disposed around the terminal end(s) of the inner side of the ocular conformer 108 in a manner configured to face one or both of the bulbar conjunctival tissues 58, 62 and/or on the terminal end(s) of the outer side of the ocular conformer 108 facing one or both of the palpebral conjunctival tissues 42, 50.

The ophthalmic medicament layer 104 may be configured to substantially cover an entire surface of the ocular conformer 108. In cases where the medicament layer 104 substantially covers the entire surface of the ocular conformer 108, the medicament layer 104 will preferably cover the inner surface of the ocular conformer 108 as illustrated in FIG. 2B. Alternatively, the ocular conformer device 100 can be configured without an inner medicament layer 104 altogether, particularly when desiring optimal venting from a device 100 using a vented ocular conformer 108.

Placement of the ophthalmic medicament layer(s) can be altered depending on the desired ocular site targeted for delivery. Thus, in contrast to methods for protecting against fibrosis in the upper and lower formices 66, 70, where the intended use is for an indication requiring, for example, corneal delivery, placement of the ophthalmic medicament layer may be restricted to the inner surface of the ocular conformer 104, including the area covering the surface of the cornea 14.

Ophthalmic medicament layers 104 can be posited onto the ocular conformer 108 materials using conventional coating methods, including spraying, immersing, and the like. For example, the a medicament solution can be applied to the ocular conformer 108 surface by either spraying the solution onto the conformer 108 or immersing the conformer 108 in the medicament solution. Whether one chooses application by immersion or application by spraying depends principally on the viscosity and surface tension of the solution; however, it is believed that spraying in a fine spray such as that available from an airbrush will provide a coating with the greatest uniformity and will provide the greatest control over the amount of coating material to be applied to the ocular conformer 108. When applying a coating by spraying or by immersion, multiple application steps may be desirable to provide improved coating uniformity and improved control over the amount of ophthalmic medicament applied to the ocular conformer 108. Methods for forming coating layers, including medicament layers, polymeric coating layers, and adhesion promoting layers are further described below.

Surface processing and/or surface activation of the base material in the ocular conformer 108 surface(s) may be employed to promote deposition and controlled release of ophthalmic medicaments, such as anti-fibrotic agents from the ophthalmic medicament layer 104. Thus, for example, ophthalmic medicaments may be incorporated into holes, wells, slots or other small apertures created in the ocular conformer base material. In such a case, deposition of the medicaments constitutes an ophthalmic medicament layer impregnated into base material of the ocular conformer 108.

Alternatively, the ophthalmic medicaments of the present invention may be directly attached to the base material of the ocular conformer 108 using ionic bonding methodologies described in U.S. Pat. Nos. 4,713,402 (Solomon) and 4,442,133 (Greco et al.), or using the covalent and/or noncovalent attachment methodologies described in U.S. Pat. Nos. 5,660,851 (Dom) and 6,063,396 (Kelleher), the disclosures of which are expressly incorporated by reference herein. Additional methods for applying ophthalmic medicaments of the present invention onto device surfaces, or impregnating medicaments within surface modified structures is described in U.S. Pat. No. 6,774,278, the disclosures of which are expressly incorporated by reference herein.

### Ophthalmic Medicaments

The ocular conformer may be modified to elute essentially any ophthalmic medicament. In a preferred embodiment, the ocular conformer is designed to elute at least one fibrosis-inhibiting agent. In a further embodiment, the ocular conformer is designed to elute at least one fibrosis-inhibiting agent in combination with a secondary therapeutic agent, such as an antibiotic. Within one embodiment of the invention, the ocular conformer is adapted to release one or more agents inhibiting any one of four general components of the fibrosis (or scarring) process, including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced. In a preferred embodiment, the anti-fibrosis agent is an antiproliferative agent or antimetabolite.

Exemplary anti-fibrosis agents for use in the ocular conformers of the present invention include: anti-proliferative agents, including taxanes, such as paclitaxel, TAXOTERE™, and docetaxel; cell cycle inhibitors, including anthracyclines, such as doxorubicin and mitoxantrone; antimetabolites, including purine analogues, such as 6-mercaptopurine, thioguanine, and pyrimidine analogues, such as 5-fluorouracil, 5-azacytidine and cytosine arabinoside, and folic acid analogues, such as methotrexate; DNA crosslinkers, such as mitomycin C; topoisomerase II inhibitors, such as etoposide; TGF-β and TGF-β receptor antagonists, including humanized anti-TGF-β2 antibodies, such as CAT-152 (lerdelimumab), snRNAs targeting TGF-β2 and the type II receptor of TGF-β (TβRII), antisense oligonucleotides or ribozymes targeting TGF-β2 or TβRII, and decorin (D-8428, Sigma-Aldrich); matrix metalloproteinase inhibitors, such as ilomastat and prinomastat; p38 MAP kinase inhibitors, such as SB202190 and SB203580; connective tissue growth factor (CTGF) antagonists; interferon-gamma; cyclosporin A; heat shock protein 90 antagonists, such as geldanamycin; inosine monophosphate dehydrogenase inhibitors, such as mycophenolic acid, 1-alpha-25 dihydroxy vitamin D3; NF-κB inhibitors; antimycotic agents, such as sulconizole; angiogenesis inhibitors; anti-scarring polypeptides and prostaglandins disclosed in U.S. Pat. Nos. 6,013,628 and 6,495,563, respectively, the disclosures of which are incorporated by reference herein; the disclosures of which are incorporated by reference herein; as well as analogues and derivatives of the aforementioned. The use of anti-proliferative agents and/or TGF-β and TGF-β receptor antagonists is particularly preferred.

Exemplary anti-proliferative agents include taxanes, such as paclitaxel, docetaxel, and TAXOTERE™; and antimetabolites, including but not limited to 5-fluorouracil, mitomycin C, methotrexate, 5-azacytidine, hydroxyurea, thiourea, 6-mercaptopurine, thioguanine, cytosine arabinoside. Taxanes, including paclitaxel, and antimetabolites, such as 5-fluorouracil are particularly preferred anti-proliferative, anti-fibrosis agents.

An anti-scarring agent may be used in combination with one or more other therapeutic agents, including additional anti-scarring agents and/or secondary ocular medicaments, wherein the combination inhibits fibrosis or scarring. An individual therapeutic component in these combinations may have anti-fibrosis or anti-scarring activity itself, or it may enhance additively or synergistically the anti-fibrosis activities of other agent(s) in the combination. The compositions of the present invention may further comprise other pharmaceutical active agents.

Secondary ocular medicaments include agents that may not have have direct anti-scarring activity or the ability to enhance the anti-scarring activity of other ocular medicaments, but may provide additional benefits associated with the anti-fibrosis treatment. Secondary ocular medicaments include, by way of example and not limitation, anti-inflammatory agents, antimicrobial agents, analgesics, ocular tissue penetration enhancers, bioadhesive or mucoadhesive polymers or agents (including as described above), and the like. Exemplary penetration enhancers include benzalkonium chloride, dimethyl sulfoxide (DMSO), decamethonium, polyoxyethylene glycol ethers, such as Brij® 35, Brij® 78, and Brij® 98, EDTA, cholates, including glycocholate and sodium taurocholate, Tween 20, bile salts, and digitonin. The selection and dose of secondary medicaments will be based on optimizing enhancing effects, while reducing irritation or other side effects.

In one embodiment, the drug eluting ocular conformer is adapted to release one or more antibiotic or antibacterial agents alone or in combination with an anti-fibrosis agent. Non-limiting examples of antibiotic agents that may be used in connection with the present invention include aminopenicillins (ampicillin, penicillin, amoxicillin, and their congeners); cephalosporins; cycloserine; macrolides (erythromycin, clarithromycin, azithromycin, roxithromycin); quinolones; rifamycins, including rifampin (RIFADIN™; RIMACTANE™); thienamycins (imipenem); tetracyclines (chlortetracycline, oxytetracycline, demeclocycline, methacycline, doxycycline, and minocycline). Additional antibiotic agents include bacitracin, cefazolin, cefdinir, cefixime, cephalexin, cefametazole, cefaclor, cefamadole, cefoxitin, cefuroxime, cefprozil, chloramphenicol, ciprofloxacin, clindamycin, clotrimazole, ethabuto, dicloxacillin, gentamycin, gramicidin, griseofulvin, itraconazole, ketoconazole, levofloxacin, loracarbef, mebendazole, metronidazole, neomycin, nitrofurantoin, nystatin, ofloxacin, polymyxin B, sparfloxacin, terbinafine, trimethoprim, tobramycin, trimethoprim, vancomycin, including analogs, and mixtures of the foregoing. Antibiotics are preferably selected to target microorganisms native to the eye area.

Non-limiting examples of synthetic antibacterial agents include sulfonamides, such as sulfacetamide, sulfadiazine, sulfamethizole, sulfaisoxazole, and sulfamethoxazole, nitrofurazone, and sodium propionate.

In a further embodiment, ocular conformers may be adapted to release one or more anti-inflammatory or immuno-suppressant agents. Non-limiting examples of anti-inflammatory agents include betamethasone, cortisone acetate, dexamethasone, dexamethasone 21-phosphate, fluorocinolone, fluoromethalone, hydrocortisone, hydrocortisone acetate, medrysone, prednisolone acetate, prednisolone 21-phosphate, rapamycin, and triamcinolone

As ocular conformers are made in a variety of configurations and sizes, the exact dose administered will vary with device size, medicament layer surface area(s) and design. However, certain principles can be applied in the application of this art. Drug dose can be calculated as a function of dose per unit area (of the portion of the device being coated), total drug dose administered, and appropriate surface concentrations of active drug can be determined. Ophthalmic medicaments may be administered to achieve sustained concentrations ranging less than 50%, less than 20%, less than 10%, less than 5%, or than 1% of the concentration conventionally used in a single, non-controlled release dose application. Preferably, the drug is released in effective concentrations for a period ranging from 1 day up to about a month or more.

### Elution Control Layer

In another aspect, a drug eluting ocular conformer of the present invention may further include additional coating layers. In a particular embodiment, a multi-layered coating includes further includes an elution control layer to further control the rate of ophthalmic medicament release and to provide a more sustained release of ophthalmic medicaments. The elution control layer may be configured as a porous layer or as bioabsorbable polymeric elastomer layer. In either case, the thickness of the elution control layer can be modified to adjust the level of release.

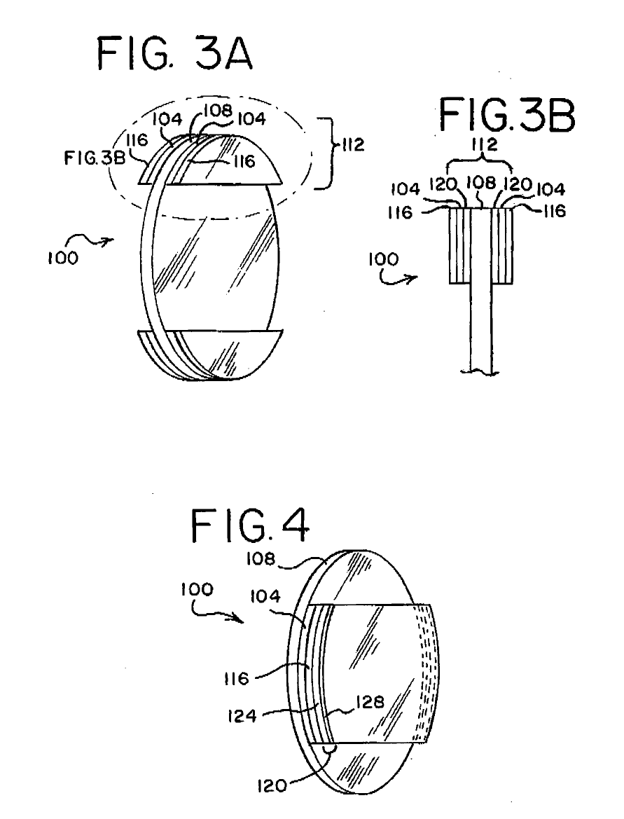

FIG. 3A shows a cross-sectional view of a coated drug-eluting ocular conformer 100 according to another embodiment illustrating an exemplary multi-layered coating 112, including an elution control layer 116. In one aspect, the elution control layer 116 includes at least one porous layer posited over at least ophthalmic medicament layer 104. In FIG. 3A, the multi-layer coating 112 includes ophthalmic medicament layer portions 104 terminally disposed on outer surface portions of the ocular conformer 108, whereby elution control layer portions 116 are positioned over corresponding ophthalmic medicament layer portions 104. Depending on the placement of the medicament layer(s) 104 as described above, elution control layer(s) 116 may be disposed in one or more locations on one side or both sides of the ocular conformer 108.

The porous layer 116 is preferably composed of a polyamide, parylene or a parylene derivative, preferably disposed on the ophthalmic medicament layer at a thickness suitable for providing controlled release of the ophthalmic medicament, preferably about 5,000 to 250,000 Å. “Parylene” is both a generic name for a known group of polymers based on p-xylylene and made by vapor phase polymerization, and a name for the unsubstituted form of the polymer; the latter usage is employed herein. More particularly, parylene or a parylene derivative is created by first heating p-xylene or a suitable derivative at an appropriate temperature (for example, at about 950° C.) to produce the cyclic dimer di-p-xylylene (or a derivative thereof. The resultant solid can be separated in pure form, and then cracked and pyrolyzed at an appropriate temperature (for example, at about 680° C.) to produce a monomer vapor of p-xylylene (or derivative); the monomer vapor is cooled to a suitable temperature (for example, below 50° C.) and allowed to condense on the desired object, for example, on the at least one layer of bioactive material. The resultant polymer has the repeating structure (CH₂C₆H₄CH₂)ₙ, with n equal to about 5,000, and a molecular weight in the range of 500,000.

When first deposited, the parylene or parylene derivative is thought to form a network resembling a fibrous mesh, with relatively large pores. As more is deposited, the porous layer 116 not only becomes thicker, but it is believed that parylene or parylene derivative is also deposited in the previously formed pores, making the existing pores smaller. Careful and precise control over the deposition of the parylene or parylene derivative therefore permits close control over the release rate of ophthalmic medicaments therethrough. It is for this reason that the ophthalmic medicaments lie under the porous layer 116, rather than being dispersed within or throughout it. Additional details concerning the deposition and configuration of porous parylene layers for controlled release of therapeutic agents, including ophthalmic medicaments according to the present invention can be found in U.S. Pat. No. 6,774,278 and U.S. Pat. Appl. No. 2007/0150047, the disclosures of which are incorporated by reference herein.

In another aspect, the elution control layer 116 may be configured as a bioabsorbable polymeric elastomer layer. The bioabsorbable elastomer layer is preferably formed from a polymer selected to provide a mechanically stable coating layer that readily recovers from deformation of the medical device without undesirable levels of irritation to surrounding tissues upon implantation. The bioabsorbable elastomer can include a hydrogel, an elastin-like peptide, a polyhydroxyalkanoates (PHA), polyhydroxybutyrate compounds, or combinations thereof. The bioabsorbable elastomer can be selected based on various design criteria, including the desired rate of release of the therapeutic agent and the degradation mechanism. In some embodiments, the bioabsorbable elastomer comprises one or more hydrolyzable chemical bonds, such as an ester, a desired degree of crosslinking, a degradation mechanism with minimal heterogeneous degradation, and nontoxic monomers.

The bioabsorbable elastomer may be a polyhydroxyalkanoate compound, a hydrogel, poly(glycerol-sebacate) or an elastin-like peptide. Desirably, the bioabsorbable elastomer includes a polyhydroxyalkanoate bioabsorbable polymer such as polylactic acid (poly lactide), polyglycolic acid (poly glycolide), polylactic glycolic acid (poly lactide-co-glycolide), poly4-hydroxybutyrate, or a combination of any of these. Preferably, the ophthalmic medicament is initially enclosed by the coating or other portions of the coated device, and does not form a portion of the external surface area of the medical device prior to release of the ophthalmic medicament.

Desirably, the bioabsorbable elastomer comprises a poly-.alpha.-hydroxy acid, such as polylactic acid (PLA). PLA can be a mixture of enantiomers typically referred to as poly-D,L-lactic acid. Alternatively, the bioabsorbable elastomer is poly-L(+)-lactic acid (PLLA) or pol-D(−)-lactic acid (PDLA), which differ from each other in their rate of biodegradation. PLLA is semicrystalline. In contrast, PDLA is amorphous, which can promote the homogeneous dispersion of an active species. Unless otherwise specified, recitation of “PLA” herein refers to a bioabsorbable polymer selected from the group consisting of: PLA, PLLA and PDLA. Preferably, the molecular weight of the bioabsorbable elastomer is about 50-500 kDa, more preferably about 60-250 kDa, and most preferably about 75-120 kDa.

The bioabsorbable elastomer can also desirably comprise polyglycolic acid (PGA). Polyglycolic acid is a simple, aliphatic polyester that has a semi-crystalline structure, fully degrades in 3 months, and can undergo strength loss within about 1 month after implantation in the body. Compared with PLA, PGA is a stronger acid and is more hydrophilic, and thus more susceptible to hydrolysis. PLA is generally more hydrophobic than PGA, and undergoes a complete mass loss in 1 to 2 years.

The bioabsorbable elastomer can also be a polylactic glycolic acid (PLGA), or other copolymers of PLA and PGA. The properties of the copolymers can be controlled by varying the ratio of PLA to PGA. For example, copolymers with high PLA to PGA ratios generally degrade slower than those with high PGA to PLA ratios. PLGA degrades slightly faster than PLA. The process of lactic acid hydrolysis can be slower than for the glycolic acid units of the PLGA co-polymer. Therefore, by increasing the PLA:PGA ratio in a PLGA co-polymer generally results in a slower rate of in vivo bioabsorption of a PLGA polymer.

Preferably, the elution control layer 116 includes or consists essentially of an amorphous poly(lactic acid) selected from the group consisting of: poly(D-lactic acid), poly(L-lactic acid) and poly(D,L-lactic acid). Increasing the amount of the biodegradable elastomer in the elution control layer 116 reduces the rate of elution of the therapeutic agent in an elution medium. Biodegradable elastomeric polymers and methods for coating such polymers are disclosed in US 2007/1096423, the disclosures of which are expressly incorporated by reference herein.

### Adhesion Promoting Layer

In a further aspect, a coated drug eluting ocular conformer device 100 of the present invention may further include at least one adhesion promoting layer 120. An adhesion promoting layer 120 can be utilized to facilitate adhesion of ophthalmic medicament layer(s) 104 to the base material of the ocular conformer 108, to ocular tissue surfaces, or both.

FIG. 3B shows a cross-sectional view of an alternative multi-layered coating 112 relative to the coating depicted in FIG. 3A, including a modified multi-layered coating 112 with adhesion promoting layers 120 disposed on the base material of the ocular conformer 108. In particular, FIG. 3B illustrates a multi-layer coating 112, including ophthalmic medicament layer portions 104 terminally disposed over the outer and inner surfaces of the ocular conformer 108 between corresponding elution control layer portions 116 and either surface of the ocular conformer 108. In this context, the adhesion promoting layers 120 are configured to facilitate adhesion to the ocular conformer 108 base material. Such an adhesion promoting layer 120 can be formed from any material suitable for promoting adhesion between the medicament layer(s) 104 and the base material of the ocular conformer 108, including but not limited to silane, pyrolytic carbon, parylene and the like. Depending on the placement of the medicament layer(s) and/or the elution control layer(s), the adhesion promoting layer(s) may be disposed in one or more locations on one side or both sides of the ocular conformer.

The multi-layered coating 112 depicted in FIGS. 3A and 3B can further include additional layers and/or combinations thereof. For example, the multilayered coating 112 in FIGS. 3A and 3B may additionally include secondary ophthalmic medicament layer(s) containing a different ophthalmic medicament. Moreover, elution control layer(s) including either a porous layer or bioabsorbable elastomeric layer may be positioned over and covering the individual ophthalmic medicament layers. The various coating layers in the multi-layer coating can have compositions and thicknesses that are the same or different. A second elution control layer may be configured to be more porous than or degrading more rapidly than the first elution control layer upon implantation. The plurality of layers in the multi-layer coating can include any suitable numbers of layers comprising the ophthalmic medicament and elution control layers, including 2, 3, 4, 5, 6, 7, 8, 9, or 10-layer coatings. Preferably, the elution control layers are positioned between the ophthalmic medicament layers. Different ophthalmic medicaments can be placed in different layers or within the same layer.

An adhesion promoting layer 120 may be further incorporated into the outside of the coated ocular drug-eluting device 100. This can allow for the ability to provide enhanced bioadhesion and delivery to ocular tissues, such as the cornea. FIG. 4 shows a cross-sectional view of an exemplary coated drug eluting ocular conformer device 100 according to a further embodiment of the present invention illustrating the incorporation of an adhesion promoting layer 120 positioned over and covering an elution control layer 116, which is positioned over and covering the medicament layer 104.

The exemplary embodiment depicted in FIG. 4 illustrates an adhesion promoting layer configured to facilitate adhesion to the corneal surface for enhanced delivery of ophthalmic medicaments thereto. Alternately, the adhesion promoting layer may be designed for better adhesion and/or delivery to subjconjunctival tissues by positioning the medicament layers to target subjconjunctival tissues as described above.

In a preferred embodiment, the adhesion promoting layer 120 includes multilayered film, including a water-soluble bioadhesive layer 124 containing at least one bioadhesive polymer and at least one water-soluble film-forming polymer, and a water-soluble non-adhesive backing layer 128. Suitable bioadhesive polymers include polyacrylic acid (PAA), which can optionally be partially crosslinked, sodium carboxymethyl cellulose (NaCMC), hydroxyethylmethyl cellulose (HEMC), hydroxypropylmethyl cellulose (HPMC), polyvinylpyrrolidone (PVP), or combinations thereof. These bioadhesive polymers are preferred because they have good and instantaneous mucoadhesive properties in a dry, film state.

Water-soluble polymer(s) of the bioadhesive layer 124 can be made from a cellulose-based polymeric derivative. Such film-forming water-soluble polymer(s) can include hydroxyethyl cellulose (HEC), hydroxypropyl cellulose (H PC), hydroxypropylmethyl cellulose (H PMC), hydroxyethylmethyl cellulose (HEMC), or a combination thereof. Similar film-forming water-soluble polymer(s) can also be used. The film-forming water-soluble polymer(s) can optionally be crosslinked and/or plasticized in order to alter its dissolution kinetics.

A water-soluble non-adhesive backing layer 128 protects the water-soluble bioadhesive layer 124. The non-adhesive backing layer 128 will dissolve after application of the adhesion promoting layer film 120 to ocular surfaces, including but not limited to conjunctival and corneal surfaces. Typically, the water-soluble non-adhesive backing layer 128 dissolves before the water-soluble bioadhesive layer 124. Dissolution of the water-soluble non-adhesive backing layer 128 primarily controls the residence time of the adhesion promoting layer 120 after application to the conjunctival and/or corneal surfaces and promotes unidirectional delivery thereto.

The water-soluble non-adhesive backing layer 128 is also water-soluble and includes pharmaceutically acceptable, water-soluble, film-forming polymer(s). The water-soluble non-adhesive backing layer 128 includes water-soluble, film-forming pharmaceutically acceptable polymer(s) including but not limited to hydroxyethyl cellulose (HEC), hydroxypropyl cellulose (H PC), hydroxypropylmethyl cellulose (H PMC), hydroxyethylmethyl cellulose (HEMC), polyvinyl alcohol (PVA), polyethylene glycol (PEG), polyethylene oxide (PEO), ethylene oxide-propylene oxide co-polymers, or a combination thereof. The water-soluble non-adhesive backing layer 128 can optionally be crosslinked. The water-soluble non-adhesive backing layer 128 can function as a slippery surface, to avoid “double-stick” to the bulbar and palpebral conjunctiva.

In one embodiment, the bioadhesive layer 124 includes a water-soluble polymer, e.g., hydroxyethylmethyl cellulose (HEMC) having both bioadhesive and film-forming capabilities. In one embodiment, the water-soluble non-adhesive backing layer 128 includes hydroxyethyl cellulose and hydroxypropyl cellulose. Other bioadhesive polymers and film-forming polymers having similarly useful properties, which are known to those of skill in the art may be used in the practice of the present invention.

Although FIG. 4 depicts an adhesion promoting layer 120 over an elution control layer 116 covering the medicament layer 104, ophthalmic medicaments could be alternatively incorporated into the bioadhesive layer 124 directly, with or without an elution control layer 120 thereover. In such a case, the bioadhesive layer 124 may be posited directly on the ocular conformer surface or indirectly by way of adhesion promoter layer therebetween.

### Coating Methods

As indicated above, the present invention contemplates the coating of one or more layers, including one or more ophthalmic medicament(s) onto an ocular conformer. The layers may be coated onto at least a portion of the ocular conformer surface facing the eyeball, at least a portion of the ocular conformer surface facing the eyelids, or both. Coating layers may be applied in sequential fashion to one or more ocular conformer surfaces. Preferably, an ophthalmic medicament layer is posited over one or both surfaces of the conformer and an elution control layer is posited over the ophthalmic medicament layer. The ophthalmic medicament(s) can be uniformly deposited over portion(s) of the two ocular conformer surface(s) and/or they may be locally deposited within holes or wells in the surface of the conformer base material. Three preferred methods for applying coating layers are described herein: (1) spray gun coating, (2) ultrasonic spray coating and (3) electrostatic spray coating.

In all three methods, a coating layer comprising a ophthalmic medicament can be formed by applying a first solution of the ophthalmic medicament to the surface of the conformer. Preferably, the first solution consists essentially of the ophthalmic medicament and a volatile solvent, and does not contain any of the above-described polymers.

In one embodiment, the ophthalmic medicament is paclitaxel and the solvent is ethanol or methanol. Desirably, a solution of about 0.5-5.0 mM paclitaxel in ethanol may used, preferably solutions of 0.7 mM, 1.2 mM paclitaxel in ethanol. Other ophthalmic medicaments and solvents may also be used in solutions at concentrations permitting desirable deposition rates forming coatings with desired durability.

After the application of the ophthalmic medicament, another layer comprising one or more polymers, such as bioabsorbable elastomers, can be dissolved in a solvent and then sprayed onto a layer of ophthalmic medicament that was previously deposited on the conformer. In one embodiment, the polymer is PLA and the solvent is dichloromethane. Desirably, about 0.1-7.0 g/L of PLA in dichloromethane is used. Even more desirably, about 2.5-6.5 g/L and most desirably 5.0 g/L of PLA in dichloromethane is used.

Each coating layer is preferably separately applied using an ultrasonic nozzle spray coating technique employing ultrasound to atomize the spray solution, to provide a smooth and uniform polymer coating. Preferably, the polymer coating is applied from an ultrasonic nozzle. A solution of about 2-4 g/L of a bioabsorbable elastomer, such as PLA in a suitable solvent such as dichloromethane can be applied using an ultrasonic nozzle. Ultrasonic nozzles can be configured such that excitation of the piezoelectric crystals creates a transverse standing wave along the length of the nozzle. The ultrasonic energy originating from the crystals located in the large diameter of the nozzle body undergoes a step transition and amplification as the standing wave as it traverses the length of the nozzle. The ultrasonic nozzle can be designed so that a nodal plane is located between the crystals. For ultrasonic energy to be effective for atomization, the atomizing surface (nozzle tip) is preferably located at an anti-node, where the vibration amplitude is greatest. To accomplish this, the nozzle's length must be a multiple of a half-wavelength. Since wavelength is dependent upon operating frequency, nozzle dimensions can be related to operational frequency. In general, high frequency nozzles are smaller, create smaller drops, and consequently have smaller maximum flow capacity than nozzles that operate at lower frequencies. The ultrasonic nozzle can be operated at any suitable frequency, including 24 kHz, 35 kHz, 48 kHz, 60 kHz, 120 kHz or higher. Preferably, a frequency of 60-120 kHz or higher is used to atomize polymer solutions to the greatest possible extent so as to promote the formation of a smooth, uniform coating. Power can be controlled by adjusting the output level on the power supply. The nozzle power can be set at any suitable level, but is preferably about 0.9-1.2 W and more preferably about 1.0-1.1 W. The nozzle body can be fabricated from any suitable material, including titanium because of its good acoustical properties, high tensile strength, and excellent corrosion resistance. Liquid introduced onto the atomizing surface through a large, non-clogging feed tube running the length of the nozzle absorbs some of the vibrational energy, setting up wave motion in the liquid on the surface. For the liquid to atomize, the vibrational amplitude of the atomizing surface can be maintained within a band of input power to produce the nozzle's characteristic fine, low velocity mist. Since the atomization mechanism relies only on liquid being introduced onto the atomizing surface, the rate at which liquid is atomized depends largely on the rate at which it is delivered to the surface. Therefore, an ultrasonic nozzle can have a wide flow rate range. The maximum flow rate and median drop diameter corresponding to particular nozzle designs can be selected as design parameters by one skilled in the art. Preferably, the flow rate is between about 0.01-2.00 mL/min, more preferably between about 0.05-1.00 and most preferably between about 0.05-0.07 mL/min.

Alternatively, the ophthalmic medicament(s) and polymer(s) can be dissolved in a solvent(s) and sprayed onto the conformer using a conventional spray gun such as a spray gun manufactured by Badger (Model No. 200), an electrostatic spray gun, or most preferably an ultrasonic nozzle spray gun. Conformer coatings comprising an ophthalmic medicament may be applied to a surface of a conformer using a spray gun. The surface of the conformer can be bare, surface modified, or a primer coating previously applied to the conformer. Preferably, the coating applied to the surface consists essentially of the ophthalmic medicament(s), and is substantially free of polymers or other materials. The ophthalmic medicaments, and optionally a polymer, can be dissolved in a solvent(s) and sprayed onto the conformer under a fume hood using a conventional spray gun, such as a spray gun manufactured by Badger (Model No. 200), or a 780 series spray dispense valve (EFD, East Providence, R.I.). Alignment of the spray gun and conformer may be achieved with the use of a laser beam, which may be used as a guide when passing the spray gun over the conformer(s) being coated.

Desirably, the ophthalmic medicament is paclitaxel and the solvent is ethanol or methanol. Desirably, a solution of paclitaxel in ethanol described above is used. The distance between the spray nozzle and the nozzle size can be selected depending on parameters apparent to one of ordinary skill in the art, including the area being coated, the desired thickness of the coating and the rate of deposition. Any suitable distance and nozzle size can be selected. For example, for coating an ocular conformer, a distance of between about 1-7 inches between the nozzle and conformer is preferred, depending on the size of the spray pattern desired. The nozzle diameter can be, for example, between about 0.014-inch to about 0.046-inch.

Varying parameters in the spray coating process can result in different solid forms of the ophthalmic medicament in a deposited coating. Spray coating parameters such as solvent system, fluid pressure (i.e., tank pressure), atomization pressure, ambient temperature and humidity. The solvent is desirably volatile enough to be readily removed from the coating during or after the spray coating process, and is preferably selected from the solvents discussed with respect to the first embodiment for each solid form of an ophthalmic medicament.

Methods of coating amorphous ophthalmic medicaments using a 780S-SS spray dispense valve (EFD, East Providence, R.I.) can comprise the steps of: dissolving solid paclitaxel in ethanol to form a solution, and spraying the solution onto a conformer with an atomization pressure of about 5-10 psi in an environment having a relative humidity of 30% or lower. Preferably, the spraying step is performed at a temperature of between about 65° F. and 75° F., and with a fluid pressure of between about 1.00 and 5.00 psi.

One or more coating layers may also be applied using an electrostatic spray deposition (ESD) process. This process is especially desirable when the ophthalmic medicament is hydrophilic. The ESD process generally depends on the principle that a charged particle is attracted towards a grounded target. The solution that is to be deposited on the target is typically charged to several thousand volts (typically negative) and held at ground potential. The charge of the solution is generally great enough to cause the solution to jump across an air gap of several inches before landing on the target. As the solution is in transit towards the target, it fans out in a conical pattern which aids in a more uniform coating. In addition to the conical spray shape, the electrons are further attracted towards the metal portions of the target, rather than towards the non conductive base the target is mounted on, leaving the coating mainly on the target only.

Generally, the ESD method allows for control of the coating composition and surface morphology of the deposited coating. In particular, the morphology of the deposited coating may be controlled by appropriate selection of the ESD parameters, as set forth in WO 03/006180 (Electrostatic Spray Deposition (ESD) of biocompatible coatings on Metallic Substrates), incorporated herein by reference. For example, a coating having a uniform thickness and grain size, as well as a smooth surface, may be obtained by controlling deposition conditions such as deposition temperature, spraying rate, precursor solution, and bias voltage between the spray nozzle and the conformer being coated. The deposition of porous coatings is also possible with the ESD method.

When spraying polymer(s) (such as PLA) onto the conformer using the ESD method, the polymer(s) are preferably dissolved in a solvent mixture comprising a mixture of dichloromethane:methanol in a 1:2 (+/−10%) ratio by volume. For example, the solvent mixture can comprise about 50-80% methanol and about 20-50% dichloromethane (by volume). More desirably, the mixture is about 65-75% methanol and about 25-40% dichloromethane (by volume). Even more desirably, the mixture is about 70% methanol and about 30% dichloromethane (by volume). It is believed that the addition of methanol to dichloromethane increases the polarity of the solvent solution, thereby providing a fine spray that is ideal for use in an electrostatic coating process. This solvent combination may provide a smooth, uniform polymer coating when applied by spraying.

### Coating Uniformity and Durability

Desirably, coatings have sufficient durability to retain a desired amount of an ophthalmic medicament after manipulations typically associated with the manufacture and delivery of the conformers to a desired point of treatment, and to function to release the ophthalmic medicament at the point of treatment at a desired rate. Durable conformer coatings preferably resist flaking, pitting or delamination as a result of physical abrasion, compression, flexion, vibration, fluid contact, and fluid shear. Conformer coatings are desirably durable enough to maintain a substantially uniform coating during sterilization and upon application at a point of treatment.

The durability of a coating can be evaluated by weighing the conformer a first time immediately after coating, subjecting the coated conformer to physical forces typical of the manufacture and delivery process for an intended use (e.g., crimping, freezing, sterilization and the like), and then weighing the coated conformer a second time. A loss in weight between the first weighing and the second weighing could indicate the loss of portions of the coating to flaking or delamination. Preferably, durable coatings for ocular conformers loose no more than about 10 μg or about 20% of the coating weight or less before and after crimping. A durable coating preferably loses less than about 15%, more preferably between about 0-10%, most preferably between about 0% and 5% of the weight of the coating during the crimping process. Durable coatings are also substantially free of “webbing,” or coating deposited over interstitial spaces between portions of a conformer.

Ocular conformers may be coated using an ultrasonic spray gun, whereby the coating layers are coated from the same solutions (e.g., a mitomycin-ethanol solution for the first layer, and a PLA-dichloromethane solution for the second layer). The coated conformers may be sterilized by a standard ethylene oxide process. The sterilization process may include subjecting the coated conformers to temperatures of about 40° C. and humidity levels of over 90%, followed by contact with ethylene oxide at about 575 mg/L for a suitable period of time to perform the sterilization. After sterilization, the coated conformers may be re-measured; the weight loss of the coating during sterilization may then be re-calculated.

Preferably, the coatings have a substantially uniform surface, without cracking or pitting. Desirably, coatings have a surface that retains surface uniformity and integrity upon sterilization. Various coating methods can be used to produce suitably smooth and durable coatings. Substantially uniform and durable coatings can be deposited by spraying a solution of a ophthalmic medicament and/or polymer(s) onto the inner and/or outer surface of a conformer using conventional pressure gun, electrostatic spray gun and ultrasonic spray gun. The uniformity of a coating can be evaluated from optical and SEM images of the surface(s).

### Methods for Delivering Ophthalmic Medicaments

In one aspect, the present invention provides a method for administering an ophthalmic medicament to the eye of a subject in which one or the above described coated drug eluting ocular conformer devices 100 is applied to an eye of a subject for the purpose of achieving a beneficial therapeutic effect. The medicament(s) and indications can include any in which topical, noninvasive drug delivery is desired. As described above, the medicament coatings can be configured to target specific ocular tissues as desired.

In a preferred embodiment, the present invention provides a method for reducing scarring in the eye in which one of the above described coated drug eluting ocular conformer devices 100 is applied to an eye of a subject. Preferably, the device 100 is applied following eye surgery or an eye injury caused by chemical burns, thermal burns, or mechanical trauma, or in conjunction with treatment of ocular diseases or conditions associated with scarring. The coated device 100 is retained in place over the eye for a period of time suitable for reducing scarring, including conjunctival scarring and/or corneal scarring.

As described above, the medicament layer(s) can be positioned to optimize targeted delivery of medicaments. In one aspect, medicament layers are configured for targeting medicament delivery to conjunctival tissues following an eye injury or an eye surgery, including but not limited to glaucoma filtration surgery and cataract surgery, where scarring around the conjunctival tissues is possible or likely if not treated. Treatment or prevention of such scarring can be further extended to various ocular diseases or conditions associated with scarring, including but not limited to recurrent ptergyia, proliferative vitreoretinopathy (PVR), Stevens-Johnson syndrome (SJS), and ocular cicatricial pemphigoid (OIP).

Alternatively, or in addition, medicament layers may be configured for targeting medicament delivery to corneal tissues for treatment and/or prevention of stromal scarring, which can be a major complication following corneal trauma, infection, or refractive surgical procedures such as radial keratotomy (RK), photorefractive keratectomy (PRK), and laser in-situ keratomileusis (LASIK).

Further, as indicated above, a coated, drug eluting ocular conformer device 100 may be used for topical delivery of ophthalmic medicaments for virtually any ophthalmic indication where controlled drug release is desirable. Thus, the present devices 100 can be used to deliver medicaments for non-scarring indications, as well, including but not limited to ocular neoplasias, such as conjunctival melanoma and primary acquired melanosis (PAM) with atypia.

A coated, drug eluting ocular conformer device 100 according to the present invention is typically configured to deliver an ophthalmic medicament in an amount sufficient to effect treatment or provide a physiologically beneficial effect when administered to a subject in need of such treatment. Such a “therapeutically effective amount” will vary depending upon the subject and disease condition being treated, the nature of the medicament and its active dosage range, the weight and age of the subject, the severity of the disease condition, the manner of administration and the like. Depending on the release kinetics of the medicament(s) and the length of time in which treatment is needed, additional coated conformer devices 100 may be reinserted as needed.

It is therefore intended that the foregoing detailed description be regarded as illustrative rather than limiting, and that it be understood that it is the following claims, including all equivalents, that are intended to define the spirit and scope of this invention.

1. A coated, drug eluting ocular conformer device comprising:
an ocular conformer formed from a base material and having inner and outer sides including apical and basal portions configured to contact one or more conjunctival tissues in an eye of a patient; and
at least one substantially purified anti-fibrosis agent, wherein the anti-fibrosis agent is formulated into at least one ophthalmic medicament layer over at least one surface of the ocular conformer or impregnated within the base material of the ocular conformer.

2. The device of claim 1, wherein the anti-fibrosis agent is impregnated within the base material of the ocular conformer.

3. The device of claim 1, wherein the medicament layer is configured to release the anti-fibrosis agent from the inner side of the ocular conformer to the upper bulbar conjunctiva and the lower bulbar conjunctiva.

4. The device of claim 3, wherein the at least one medicament layer is configured not to release the anti-fibrosis agent from the outer side of the ocular conformer to the cornea.

5. The device of claim 1, wherein the at least one medicament layer is configured to release the anti-fibrosis agent to the cornea, but not to the inner side of the ocular conformer to the upper bulbar conjunctiva and the lower bulbar conjunctiva.

6. The device of claim 1, wherein the at least one medicament layer is configured to release the anti-fibrosis agent from the outer side of the ocular conformer to the upper palpebral conjunctiva and the lower palpebral conjunctiva.

7. The device of claim 4, comprising medicament layers configured to release the anti-fibrosis agent from both sides of the ocular conformer to each of the upper bulbar conjunctiva, lower bulbar conjunctiva, upper palpebral conjunctiva, and lower palpebral conjunctiva.

8. The device of claim 1, wherein the at least one medicament layer is formed by spraying the anti-fibrosis agent onto at least one surface of the ocular conformer.

9. The device of claim 1, further comprising an elution control layer posited over the at least one medicament layer or over the base material impregnated with the anti-fibrosis agent.

10. The device of claim 9, wherein the elution control layer is comprised of a porous polymer.

11. The device of claim 10, wherein the porous polymer comprises a parylene or a parylene derivative.

12. The device of claim 9, wherein the elution control layer is comprised of a biodegradable elastomeric polymer.

13. The device of claim 12, wherein the biodegradable elastomer comprises a polylactic acid, a polyglycolic acid, or a copolymer therefrom.

14. The device of claim 1, further comprising an adhesion promoting layer.

15. The device of claim 14, wherein the adhesion promoting layer is between the medicament layer and a surface of the ocular conformer.

16. The device of claim 14, wherein the adhesion promoting layer comprises a multilayer coating on an outer surface on the inner side of the ocular conformer.

17. The device of claim 16, wherein the multilayer coating comprises a bioadhesive layer and a water-soluble non-adhesive backing layer, wherein the bioadhesive layer comprises at least one bioadhesive polymer and at least one water-soluble film-forming polymer.

18. The device of claim 17, wherein the bioadhesive polymer is selected form the group consisting of polyacrylic acid, sodium carboxymethyl cellulose, hydroxyethylmethyl cellulose, hydroxypropylmethyl cellulose, polyvinylpyrrolidone (PVP), and combinations thereof.

19. A method for making a coated, drug eluting ocular conformer device according to claim 1 comprising:
providing an ocular conformer formed from a base material, the conformer having an inner side and an outer side, the inner side being configured to contact an eye; and
impregnating within the base material of the ocular conformer at least one substantially purified anti-fibrosis agent or positing on at least one side of the ocular conformer an ophthalmic medicament layer comprising a substantially purified anti-fibrosis agent.

20. A method for protecting against scarring in the eye comprising applying to an eye of a patient, the coated, drug eluting ocular conformer device according to claim 1.
